(19) United States
(12) Patent Application Publication
(10) Pub. No.: US 2025/0177696 A1
(43) Pub. Date: Jun. 5, 2025

(54) CAHETERS FOR LOCO-REGIONAL PERFUSION SYSTEMS

(71) Applicant: DINAQOR AG, Schlieren (CH)

(72) Inventors: Johannes HOLZMEISTER, London (GB); Valeria RICOTTI, London (GB); Mark DEHDASHTIAN, Irvine, CA (US); Max EMMERT, Ebmatingen (CH); Nikola CESAROVIC, Zürich (CH); Ole BLANK, Zürich (CH); Nadine TROES, Bülach (CH); Philippe Bennani KAMANE, La Croix Sur Lutry (CH)

(21) Appl. No.: 18/839,215

(22) PCT Filed: Feb. 17, 2023
(86) PCT No.: PCT/EP2023/054105

§ 371 Date: Aug. 16, 2024

Related U.S. Application Data
(60) Provisional application No. 63/312,029, filed on Feb. 20, 2022.

(51) Int. Cl. A61M 25/00; A61M 25/04; A61M 25/10; A61M 27/00

(52) U.S. Cl. CPC A61M 25/007; A61M 25/1011; A61M 27/00; A61M 2025/0039; A61M 2025/0079; A61M 25/04; A61M 2025/1052; A61M 2205/32; A61M 2210/125

(57) ABSTRACT

Disclosed herein are catheters (100) designed for improved performance in loco-regional perfusion systems, including perfusion catheters for a supply line and recovery catheters for a return line. In certain embodiments, the catheters include an occlusion structure (110). In certain embodiments, the catheters are configured to support a liquid flow rate of about 400 mL/min or greater.

### CROSS-REFERENCE TO RELATED APPLICATION(S)

This application claims the benefit of priority of U.S. Provisional App. No. 63/312,029, filed on Feb. 20, 2022, the disclosure of which is hereby incorporated by reference herein in its entirety.

### FIELD OF THE INVENTION

The present invention relates to catheters, particularly, catheters designed for improved performance in a loco-regional perfusion system.

### BACKGROUND

Gene therapy and cell therapy techniques in the treatment have attracted increased attention due to their potential to be uniquely tailored and efficacious in addressing the root cause pathogenesis of various conditions. Nevertheless, issues related to delivery, including vector efficiency, dose, specificity, and safety remain. As such, there is a need for further research directed to ways of achieving a more targeted, homogenous delivery of drugs suitable for treatment of various conditions. In addition, there is a need for improved devices to facilitate local delivery of gene therapy drugs into the body to treat target organs, for example, by isolating the target organs from the systemic circulation.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 illustrates an exemplary recovery catheter having a lumen shaft with a proximal end and a distal end in accordance with at least one embodiment;

FIG. 2 is an image of a catheter with a balloon in its deployed state in accordance with at least one embodiment;

FIG. 3 illustrates insertion of an exemplary catheter into the coronary sinus via the right atrium according to at least one embodiment;

FIG. 4 illustrates a catheter that is only partially inserted into the coronary sinus such that it abuts the ostium of the coronary sinus in accordance with at least one embodiment;

FIG. 5 illustrates the use of a two-catheter system according to at least one embodiment;

FIG. 6 illustrates a variant of the two-catheter system according to at least one embodiment;

FIG. 7 illustrates the use of a single catheter that includes multiple balloons according to at least one embodiment;

FIG. 8 illustrates a catheter that includes a partially covered and recapturable stent structure according to at least one embodiment;

FIG. 9 illustrates a catheter that includes a deployable and retractable stent structure according to at least one embodiment;

FIG. 10 illustrates a catheter that includes a covered disk-shaped stent structure according to at least one embodiment;

FIG. 11A illustrates a first exemplary perfusion catheter having a deployable balloon according to at least one embodiment;

FIG. 11B illustrates the first exemplary perfusion catheter with a balloon in its deployed state according to at least one embodiment;

FIG. 11C illustrates the first exemplary perfusion catheter with the balloon in its deflated state according to at least one embodiment;

FIG. 11D illustrates deployment of the first exemplary perfusion catheter in an aorta according to at least one embodiment;

FIG. 12A illustrates a second exemplary perfusion catheter having a plug according to at least one embodiment;

FIG. 12B illustrates the plug of the second exemplary perfusion catheter in a retracted state according to at least one embodiment;

FIG. 12C illustrates the plug of the second exemplary perfusion catheter in an extended state according to at least one embodiment;

FIG. 12D illustrates deployment of the second exemplary perfusion catheter in an aorta according to at least one embodiment;

FIG. 13A illustrates a third exemplary perfusion catheter having a wedge according to at least one embodiment;

FIG. 13B illustrates the wedge of the third exemplary perfusion catheter according to at least one embodiment;

FIG. 13C illustrates a further view of the wedge of the third exemplary perfusion catheter according to at least one embodiment;

FIG. 13D illustrates deployment of the third exemplary perfusion catheter in an aorta according to at least one embodiment;

FIG. 14A illustrates a fourth exemplary perfusion catheter that includes a partially covered and recapturable sent structure according to at least one embodiment;

FIG. 14B illustrates placement of the stent structure of the fourth exemplary perfusion catheter according to at least one embodiment;

FIG. 14C illustrates deployment of the stent structure of the fourth exemplary perfusion catheter according to at least one embodiment;

FIG. 15A illustrates a fifth exemplary perfusion catheter that includes a releasable covered braided disk according to at least one embodiment;

FIG. 15B illustrates the releasable covered braided disk of the fifth exemplary perfusion catheter in a deployed state according to at least one embodiment;

FIG. 16A illustrates a sixth exemplary perfusion catheter according to at least one embodiment;

FIG. 16B illustrates placement of the sixth exemplary perfusion catheter according to at least one embodiment;

FIG. 16C illustrates deployment of the sixth exemplary perfusion catheter in an aorta according to at least one embodiment;

FIG. 16D illustrates a pre-shaped catheter lumen for the sixth exemplary perfusion catheter according to at least one embodiment; and

FIG. 17 illustrates preformed lumen shafts in accordance with various embodiments.

### SUMMARY

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

One aspect of the present disclosure relates to a recovery catheter comprising: a lumen shaft having a proximal end and a distal end; an expandable balloon structure disposed near the distal end of the lumen shaft; and a tip portion between the balloon structure and the distal end of the lumen shaft. In at least one embodiment, the recovery catheter is configured to support a liquid flow rate of about 150 mL/min or greater, or about 400 mL/min or greater.

In at least one embodiment, the tip portion comprises an elongated shaft extending from the balloon portion to the distal end of the lumen shaft. In at least one embodiment, the tip comprises a distal opening at the distal end and a plurality of perforations along the elongated shaft. In at least one embodiment, the length of the elongated shaft of the tip portion is from about 2 mm to about 35 mm, about 5 mm to about 30 mm, about 10 mm to about 25 mm, or about 15 mm to 25 mm.

In at least one embodiment, an inner diameter of the lumen shaft is at least about 2 mm.

In at least one embodiment, an expanded diameter of the balloon structure is from about 15 mm to about 30 mm, about 15 mm to about 20 mm, about 20 mm to about 25 mm, about 24 mm to about 28 mm, or about 25 mm to about 30 mm.

In at least one embodiment, the lumen shaft is a multi-lumen shaft comprising at least two lumen shafts.

In at least one embodiment, the multi-lumen shaft comprises an inner lumen shaft and an outer lumen shaft that at least partially encompasses the inner lumen shaft to expose a proximal portion of the inner lumen shaft near the proximal end of the lumen shaft. In at least one embodiment, the balloon structure and the tip portion are disposed on the inner lumen shaft.

In at least one embodiment, the recovery catheter is a multi-balloon catheter further comprising at least an additional expandable balloon structure disposed adjacent to the primary balloon structure. In at least one embodiment, an expanded diameter of the additional balloon structure is greater than an expanded diameter of the primary balloon structure. In at least one embodiment, a portion of the lumen shaft between the balloon structures comprises one or more perforations.

In at least one embodiment, the recovery catheter further comprises: a stent structure disposed on a portion of the lumen shaft between the tip portion and the balloon structure.

In at least one embodiment, the recovery catheter further comprises: perforations along the lumen shaft between the balloon structure and the stent structure and/or between the stent structure and the distal end.

Another aspect of the present disclosure relates to a recovery catheter comprising: a lumen shaft having a proximal end and a distal end; a covered disk-shaped stent structure disposed near the distal end of the lumen shaft; and a tip portion between the covered disk-shaped stent structure and the distal end of the lumen shaft. In at least one embodiment, the recovery catheter is configured to support a liquid flow rate of about 150 mL/min or greater, or about 400 mL/min or greater.

In at least one embodiment, the tip portion comprises a radio marker or a radiopaque filler composition.

Another aspect of the present disclosure relates to a recovery catheter comprising: a lumen shaft having a proximal end and a distal end; and a deployable stent structure disposed at the distal end of the lumen shaft, the stent structure comprising a covering on a proximal portion. In at least one embodiment, the stent structure, when deployed, is uncovered at a distal portion. In at least one embodiment, the recovery catheter is configured to support a liquid flow rate of about 150 mL/min or greater, or about 400 mL/min or greater.

In at least one embodiment, any of the foregoing recovery catheters are configured to support a liquid flow rate of at least about 150 mL/min, at least about 200 mL/min, at least about 250 mL/min, at least about 300 mL/min, at least about 350 mL/min, at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, or at least about 1000 mL/min. In at least one embodiment, any of the foregoing catheters are configured to support a liquid flow rate of about 150 mL/min to about 1000 mL/min.

Another aspect of the present disclosure relates to a perfusion catheter comprising: a lumen shaft having a proximal end and a distal end; an occlusion structure near the distal end of the lumen shaft, the occlusion structure adapted to seal perfused blood from systemic blood circulation when inserted into a vascular blood vessel; and a tip portion comprising an elongated shaft extending from the occlusion structure to the proximal end of the lumen shaft, the tip comprising a proximal opening at the proximal end. In at least one embodiment, the occlusion structure is selected from: a balloon; a plug configurable between a retracted state and an extended state; a wedge shaped to adapt to a vessel or ostium; a releasable covered stent; and a releasable braided disk. In at least one embodiment, the perfusion catheter is configured to support a liquid flow rate of about 150 mL/min or greater, or about 400 mL/min or greater.

In at least one embodiment, the occlusion structure comprises the plug configurable between a retracted state and an extended state. In at least one embodiment, when in the extended state, a portion of the plug extends distally from the tip portion.

In at least one embodiment, the occlusion structure comprises the releasable covered stent. In at least one embodiment, the lumen shaft comprises an outer sheath that covers the releasable covered stent. In at least one embodiment, when the sheath is retracted proximally, the releasable covered stent is deployed past the distal end of the lumen shaft having an expanded diameter that is greater than the diameter of the lumen shaft.

In at least one embodiment, the occlusion structure comprises the releasable braided disk. In at least one embodiment, the lumen shaft comprises an outer sheath that covers the releasable braided disk. In at least one embodiment, when the sheath is retracted proximally, the releasable braided disk is deployed having an expanded diameter that is greater than the diameter of the lumen shaft. In at least one embodiment, the tip portion extends distally past the releasable braided disk when deployed.

In at least one embodiment, any of the foregoing perfusion catheters are configured to support a liquid flow rate of at least about 150 mL/min, at least about 200 mL/min, at least about 250 mL/min, at least about 300 mL/min, at least about 350 mL/min, at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, or at least about 1000 mL/min.

### Definitions

As used herein, the singular forms “a,” “an,” and “the” include plural references unless the context clearly indicates otherwise. Thus, for example, reference to “a drug” includes a single drug as well as a mixture of two or more different drugs.

Also as used herein, “about,” when used in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

Also as used herein, “perfusion,” “perfused,” and “perfusing” have their ordinary and customary meaning in the art and refer to administration for a time period (typically a minute or more) that is substantially longer than the art recognized term of “injection” or “bolus injection” (typically less than a minute). The flow rate of the perfusion will depend at least in part on the volume administered.

Also as used herein, “isolated,” “substantially isolated,” “largely isolated,” and their variants are terms that do not require complete or absolute isolation of the renal or systemic circulation; rather, they are intended to mean that a majority, preferably the major part or even substantially all of the specified circulation is isolated. Also as used herein, “partially isolated” refers to any nontrivial portion of the specified circulation being isolated.

Also as used herein, “non-naturally restricted” includes any method of restricting the flow of fluid through a blood vessel, e.g., balloon catheter, sutures, etc., but does not include naturally occurring restriction, e.g., plaque build-up (stenosis). Non-natural restriction includes substantial or total isolation of, for example, the renal circulation.

Also as used herein, “minimally invasive” is intended to include any procedure that does not require open surgical access to the kidney or vessels closely associated with the kidney. Such procedures include the use of endoscopic means to access the kidney, and also catheter-based means relying on access via large arteries and veins.

Also as used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

Also as used herein, a “subject” encompasses the definition of the term “patient” and does not exclude individuals who are otherwise healthy.

Also as used herein, “treatment of” and “treating” include the administration of a drug with the intent to lessen the severity of or prevent a condition, e.g., a renal condition or renal disease.

Also as used herein, an “active agent” refers to any material that is intended to produce a therapeutic, prophylactic, or other intended effect, whether or not approved by a government agency for that purpose.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

Certain embodiments of the present disclosure are directed to catheters for use in loco-regional perfusion (LRP) systems. The catheters are designed for high liquid flow rates through organs in which LRP is to be performed, while providing improved sealing and isolation of the LRP circuit and stability for catheter positioning during perfusion and recovery of the perfusate.

LRP may be performed in various bodily organs including, but not limited to, the heart, the kidneys, and the liver. Exemplary systems to perform an LRP procedure in an unarrested beating heart are described in International Application No. PCT/IB2020/000692, filed Aug. 26, 2020, and International Application No. PCT/EP2022/054361, filed Feb. 22, 2022, the disclosures of which are hereby incorporated by reference herein in their entireties. The coronary circulation provides blood supply to the tissue of the heart, in which there are a number of coronary arteries. Normally, four main coronary arteries provide oxygenated blood to the heart for distribution throughout the heart tissue: the left main and right coronary arteries, the left anterior descending artery, and the left circumflex artery. Oxygen depleted blood flows through the coronary sinus.

An exemplary LRP procedure for the heart involves isolating the coronary circulation of a patient from the systemic circulation of the patient by forming a closed circuit through the coronary circulation that includes a first perfusion catheter connected to a supply line, a second perfusion catheter connected to the supply line, a recovery catheter connected to a return line, and an external membrane oxygenation device. In an exemplary system, each perfusion catheter is inserted into a coronary artery, and the recovery catheter is inserted into the coronary sinus. A perfusate comprising a drug suitable for treatment of a heart condition can be delivered to the heart muscle while substantially isolating the patient's coronary circulation from the patient's systemic circulation with the closed circuit.

An exemplary LRP procedure for the kidney involves positioning a perfusion catheter in the renal artery of the kidney and positioning a recovery catheter in the renal vein of the kidney. A closed circuit is created by the perfusion catheter and the recovery catheter in combination with a membrane oxygenation device. A perfusate is then flowed through the closed circuit in the renal circulation, which is substantially isolated from the systemic circulation of the patient. An additional recovery catheter can be inserted into the bladder to measure urine excretion during the perfusion. An exemplary system to perform an LRP procedure in a kidney is described in International Application No. PCT/EP2022/054360, filed Feb. 22, 2022, the disclosure of which is hereby incorporated by reference herein in its entirety.

An exemplary LRP procedure for the liver involves positioning a first perfusion catheter in the hepatic artery, positioning a second perfusion catheter in the portal vein, and positioning one or more recovery catheters in the inferior vena cava proximal to the liver. A closed circuit is created by the perfusion catheters and the one or more recovery catheters in combination with one or more membrane oxygenation devices. A perfusate is then flowed through the closed circuit in the hepatic circulation, which is substantially isolated from the systemic circulation of the patient. An exemplary system to perform an LRP procedure in a liver is described in International Application No. PCT/EP2022/054356, filed Feb. 22, 2022, the disclosure of which is hereby incorporated by reference herein in its entirety.

Exemplary recovery catheters and perfusion catheters are now described. While the various catheter embodiments are depicted as being deployed within the heart, it is to be understood that deployment in the heart is merely illustrative. The catheters can be configured for the anatomy of the target organ for which LRP is to be performed, as would be appreciated by those of ordinary skill in the art. Moreover, it is to be understood that any of the catheters described as “recovery catheters” could also be used as “perfusion catheters,” and vice versa. The embodiments described herein are not limited to LRP of a target organ, but may also be used to isolate the circulation of the target organ from the systemic circulation, for example, to reduce or prevent exposure of the target organ to a drug or other agent introduced into the systemic circulation that may have a deleterious effect on the target organ. Those of ordinary skill in the art would appreciate other uses of the catheter embodiments described herein, for example, in applications for which sealing of a blood vessel is desired.

### Recovery Catheter Embodiments

Embodiments of exemplary catheters for use as recovery catheters in an LRP system are now described. In at least one embodiment, the recovery catheters are designed to support a liquid suction flow rate of about 150 mL/min or greater (e.g., about 700 mL/min or greater). For example, in certain embodiments, an exemplary catheter can support an in vitro suction flow rate of about 800 mL/min at about −80 mmHg.

Certain embodiments of the recovery catheters are advantageous for use in the return line of an LRP system used to form a closed-circuit within an unarrested beating heart when inserted into the coronary sinus. The catheters described herein can be designed to satisfy the following criteria: capability to access the coronary sinus via the right internal jugular vein; compatibility with an introducer sheath having an inner diameter of 24 Fr or less; compatibility with a 0.035-inch guidewire or smaller; capability to access, seal, and occlude a coronary sinus having a vessel internal diameter of 6 to 20 mm in a human subject or up to 30 mm in a porcine animal model; the ability to avoid occlusion of prominent side veins (e.g., the middle cardiac vein); and the ability to maintain stable position for at least 60 minutes during an LRP procedure.

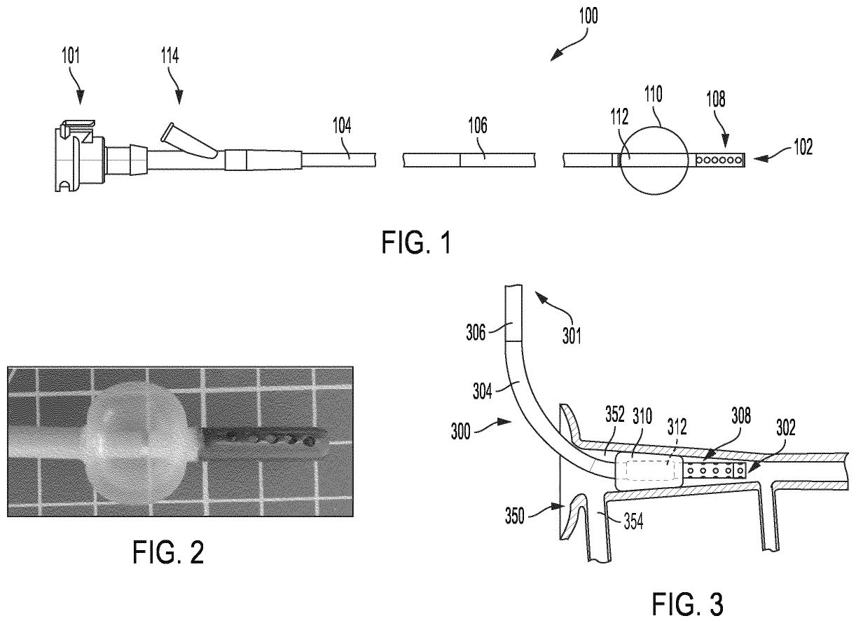

FIGS. 1-10 depict various catheter embodiments suitable for fluid recovery in an LRP system. Any of the catheters depicted in FIGS. 1-10 may be configured to support liquid flow rates (suction or perfusion) of at least about 150 mL/min, at least about 200 mL/min, at least about 250 mL/min, at least about 300 mL/min, at least about 350 mL/min, at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, at least about 1000 mL/min, or within any range defined by any of the foregoing lower limits (e.g., from about 150 mL/min to about 1000 mL/min, from about 400 mL/min to about 800 mL/min, etc.). Each catheter may be be compatible with a stearable introducer sheath, which provides stability and directs the distal end of the catheter, and allows for the catheter to create a directed push force. Each catheter may also have a pull wire integrated into its shaft assembly, allowing for sections proximal to the occlusion structure to bend at angles of up to 120° and achieve better tracking and centering of the occlusion structure.

In certain embodiments, one or more of the catheters may be multi-lumen catheters, such as double-lumen catheters. In certain embodiments, the multi-lumen catheters allow for liquid flow (e.g., a perfusate) and enable inflation of one or more balloons. In certain embodiments, one or more of the catheters may be multi-balloon catheters having two or more balloons. In certain embodiments, one or more of the balloons may be deployed or deflated independently.

FIG. 1 illustrates an exemplary catheter 100 having a lumen shaft 104/106 with a proximal end 101 and a distal end 102. The lumen shaft 104/106 can be formed from an outer lumen shaft 104 that at least partially encompasses an inner lumen shaft 106 to expose a distal portion of the inner lumen shaft 106 near the distal end 102. The proximal end 101 includes an outlet structure that can be fluidly coupled to an LRP system. One or more of the outer lumen shaft 104 or the inner lumen shaft 106 may be formed from a durable polymer material such as a polyether block amide (PEBA) material (e.g., commercially available as PEBAX®). In at least one embodiment, an innermost diameter (“inner diameter”) of the inner lumen shaft 106 is at least about 4 mm to provide a liquid flow path. In at least one embodiment, the catheter 100 may be designed to include additional lumen shafts.

The catheter 100 includes a tip portion 108 at the distal end 102 and an expandable balloon structure 110 disposed along a portion 112 of the inner lumen shaft 106. In at least one embodiment, the tip portion 108 includes an elongated shaft extending from the balloon structure 110 to the distal end 102. In at least one embodiment, the length of the elongated shaft of the tip portion is from about 2 mm to about 35 mm, about 5 mm to about 30 mm, about 10 mm to about 25 mm, about 15 mm to 25 mm, or within any subrange defined between (e.g., about 2 mm to about 5 mm). In at least one embodiment, the tip portion 108 includes an opening at the distal end 102 and one or more perforations along the elongated shaft. In at least one embodiment, the tip portion is formed from a compliant material that is more flexible than the material of the inner lumen shaft 106.

In at least one embodiment, the inner lumen shaft 106 includes a concentric inner flow path surrounding the liquid flow path. The concentric inner flow path provides a path for gas flow from the balloon structure 110 to a port 114, which can be used to inflate or deflate the balloon depending on the pressure applied at the port 114. In at least one embodiment, an outermost surface of the inner lumen shaft 106 at the portion 112 is removed such that the portion 112 is sealed by the balloon structure 110 to isolate gas flow from the concentric inner flow path to the balloon structure 110. In at least one embodiment, an expanded diameter of the balloon structure is from about 15 mm to about 30 mm, about 15 mm to about 20 mm, about 20 mm to about 25 mm, about 24 mm to about 28 mm, or about 25 mm to about 30 mm.

FIG. 2 is an image of a catheter having a similar structure to the catheter 100 with a balloon in its deployed state. The dimensions of the catheter include: a crossing profile of 19 Fr (6.3 mm); an innermost diameter of 12 Fr (4.0 mm); a usable length of 80 cm; a balloon diameter (when deployed) of 25 mm; and a tip portion length of 20 mm. The lumen shaft can be formed from a polymer material such as PEBAX® 63 that is supported by a strong stainless-steel braid. The balloon can be formed from a compliant thermoplastic/elastomeric material such as ChronoPrene™ 25A. The tip portion can be formed from a polymer material such as PEBAX® 35 and can be loaded with a radio marker or a radiopaque filler composition, such as BaSO₄.

FIG. 3 illustrates insertion of an exemplary catheter 300 into the coronary sinus 352 via the right atrium 350 according to at least one embodiment. The catheter 300 may be the same as or similar to the catheter 100, having a proximal end 301, a distal end 302, an inner lumen shaft 304, an outer lumen shaft 306, a tip portion 308, and a balloon structure 310 disposed on a portion 312 of the inner lumen shaft 304. The balloon structure 310 when deployed is compliant enough to adapt to the anatomy of the coronary sinus 352 and occlude the blood flow through the coronary sinus 352 into the right atrium 350 without creating excessive force on the tissue. As illustrated in FIG. 3, the catheter 300 is inserted past the middle cardiac vein (MCV) 354 so as to avoid occluding the flow from the MCV 354 into the atrium 350.

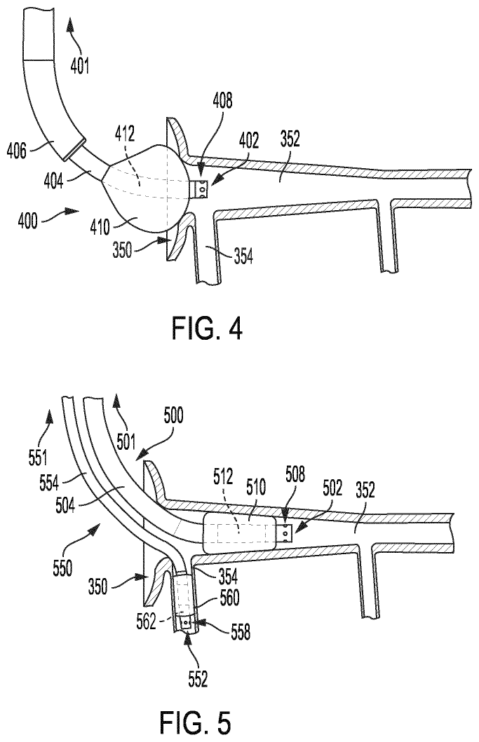

FIGS. 4-10 illustrate other occlusion techniques in accordance with various embodiments of the disclosure. The catheters depicted in FIGS. 4-10 may be similar in certain aspects to the catheters depicted in FIGS. 1-3, for example, in terms of dimensions, materials, or structures.

FIG. 4 illustrates a catheter 400 according to at least one embodiment that is only partially inserted into the coronary sinus 352 such that it abuts the ostium of the coronary sinus 352. The catheter 400 includes a proximal end 401, a distal end 402, an inner lumen shaft 404, an outer lumen shaft 406, a tip portion 408, and a balloon structure 410 disposed on a portion 412 of the inner lumen shaft 404. In at least one embodiment, a diameter of the balloon structure 410 is greater than about 15 mm, greater than about 20 mm, greater than about 25 mm, or greater than about 30 mm when deployed. The tip portion 408 may include, in addition to an opening at the distal end 402, one or more perforations to facilitate flow of blood from the coronary sinus 352 and the MCV 354 into the catheter 400.

In at least one embodiment, during deployment, the outer lumen shaft 406 can be moved distally to abut against the deployed balloon structure 410, resulting in additional pressure by the balloon structure 410 against the ostium of the coronary sinus 352 to further stabilize the position of the catheter 400. In at least another embodiment, a wire structure may be utilized to apply pressure to the balloon structure 410. The wire structure, for example, may have a sinusoidal shape that is deployable to an expanded flower-like structure extending radially from the outer lumen shaft 406 or the inner lumen shaft 404. When brought into contact with the balloon structure 410, the wire structure may produce a more even pressure profile across the surface of the balloon structure 410. Prior to deployment, the wire structure may be covered by the outer lumen 406, or may be covered by an additional lumen outside of the outer lumen 406.

FIG. 5 illustrates the use of a first catheter 500 and a second catheter 550 for separately occluding and draining the coronary sinus 352 and the MCV 354, respectively, according to at least one embodiment. The first catheter 500 includes a proximal end 501, a distal end 502, a lumen shaft 504, a tip portion 508, and a balloon structure 510 disposed on a portion 512 of the lumen shaft 504. Similarly, the second catheter 550 includes a proximal end 551, a distal end 552, a lumen shaft 554, a tip portion 558, and a balloon structure 560 disposed on a portion 562 of the lumen shaft 554. In this configuration, the first catheter 500 is inserted into the coronary sinus 352 such that the balloon structure 510 does not occlude the MCV 354, while the second catheter 550 is inserted directly into the MCV 354. The dimensions of the first catheter 500 and the second catheter 550 may be selected to provide safe and effective occlusion of the coronary sinus 352 and the MCV 354, respectively.

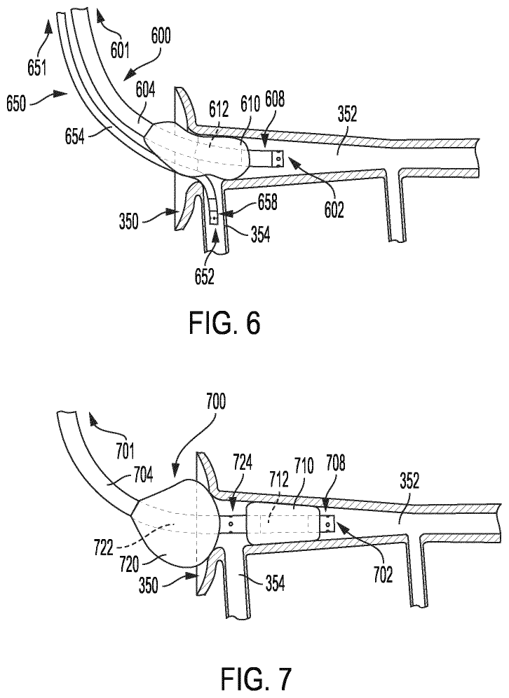

FIG. 6 illustrates a variation of FIG. 5, which uses two catheters with only one having a balloon structure according to at least one embodiment. A first catheter 600 includes a proximal end 601, a distal end 602, a lumen shaft 604, a tip portion 608, and a balloon structure 610 disposed on a portion 612 of the lumen shaft 604. A second catheter 650 includes a proximal end 651, a distal end 652, a lumen shaft 654, and a tip portion 658, and does not include a balloon structure. The first catheter 600 is inserted into the coronary sinus 352 such that a portion of the balloon 610 occludes the MCV 354 and is partially within the atrium 350 and the coronary sinus 352. The catheter 650 is inserted directly into the MCV 354 and is disposed between the vessel wall and the balloon 610, which at least partially occludes the MCV 354.

FIG. 7 illustrates the use of a single catheter 700 that includes multiple balloons according to at least one embodiment. The catheter 700 includes a proximal end 701, a distal end 702, a lumen shaft 704, a tip portion 708, a first balloon structure 710 disposed on a first portion 712 of the lumen shaft 704, and a second balloon structure 720 disposed on a second portion 722 of the lumen shaft 704. In at least one embodiment, the catheter 700 is designed for insertion into the coronary sinus 352 such that the first balloon structure 710 occludes the coronary sinus 352, and the second balloon structure 720 abuts the ostium of the coronary sinus 352 to occlude the MCV 354 (and further occlude the coronary sinus 352). An intermediate portion 724 of the lumen shaft 704 between the first balloon structure 710 and the second balloon structure 720 includes one or more perforations to allow drainage of the MCV 354. In at least one embodiment, an expanded diameter of the second balloon structure 720 is greater than an expanded diameter of the first balloon structure 710. In at least one embodiment, the catheter 700 is a multi-lumen catheter designed to allow each balloon to be deployed and deflated independently of each other.

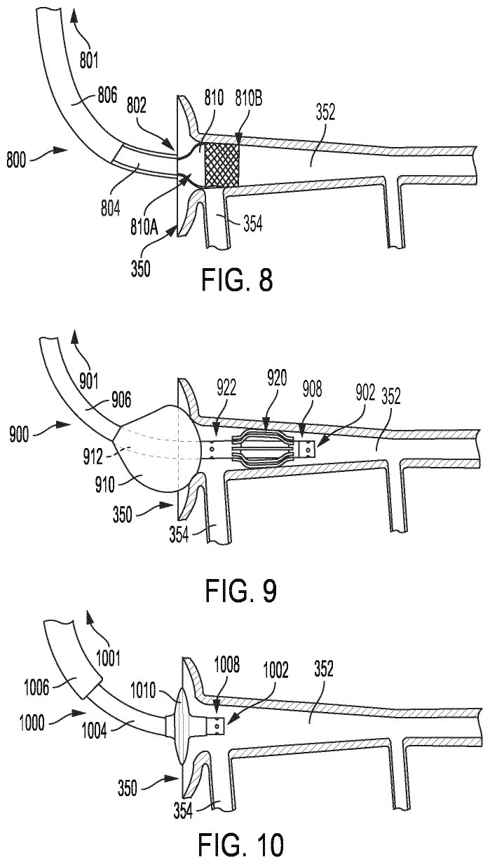

FIG. 8 illustrates a catheter 800 that includes a partially covered and recapturable stent structure 810 according to at least one embodiment. The catheter 800 includes a proximal end 801 and a distal end 802, an inner lumen shaft 804 coupled to the stent structure 810, and an outer lumen shaft 806. Part of the outer lumen shaft 806 is depicted as a cutaway view to illustrate the inner lumen shaft 804 within. The stent structure 810 is depicted in its deployed state, but can be contained within the outer lumen shaft 806 prior to deployment. The stent structure 810 is further depicted as having a proximal covered portion 810A, which may be formed from a flexible and durable polymer material, and an distal uncovered portion 810B. When inserted into the coronary sinus 352, as shown, the covered portion 810A occludes blood flow out of the coronary sinus 354, while the uncovered portion 810A provides structural support within the coronary sinus 354 while allowing blood flow from both the coronary sinus 352 and the MCV 354 directly into the catheter 800. In at least one embodiment, the catheter 800 can be used as a perfusion catheter connected to a supply line.

FIG. 9 illustrates a catheter 900 that includes a deployable and retractable stent structure 920 according to at least one embodiment. The catheter 900 further includes a proximal end 901, a distal end 902, a lumen shaft 906, a tip portion 908, and a balloon structure 910 disposed on a portion 912 of the lumen shaft 906. The catheter 900 can further include an outer lumen shaft (not shown) that substantially encapsulates the stent structure 920 and the balloon structure 910 prior to deployment. Deployment of the stent structure 920 can be performed by moving the outer lumen shaft in a proximal direction, and retraction of the stent structure 920 can be performed by moving the outer lumen shaft in a distal direction. The stent structure 920 may be formed from, for example, stainless-steel, and is disposed between the balloon structure 910 and the tip portion 908. In at least one embodiment, the lumen shaft 906 comprises at least one perforation along a portion 922 between the balloon structure 910 and the stent structure 920 to allow drainage of the MCV 354 into the catheter 900. When inserted into the coronary sinus 352, The balloon structure 910 abuts the ostium of the coronary sinus 352.

FIG. 10 illustrates a catheter 1000 that includes a covered disk-shaped stent structure 1010 according to at least one embodiment. The catheter 1000 further includes a proximal end 1001, a distal end 1002, an outer lumen shaft 1006, an inner lumen shaft 1004, and a tip portion 1008. The stent structure 1010 may be formed from, for example, a stainless-steel stent having a durable polymer covering. The outer lumen shaft 1006 can cover the stent structure 1010 prior to deployment. Once the catheter 1000 is properly positioned, the outer lumen shaft 1006 can be moved in the proximal direction to enable deployment of the stent structure 1010. In at least one embodiment, the stent structure 1010 is coupled to the tip portion 1008, which may be partially contained within the inner lumen shaft 1004 and can be actuatable (using a wire) to deploy the stent structure 1010 when moved in a proximal direction and retract the stent structure 1010 when moved in a distal direction. In at least one embodiment, the stent structure 1010, when deployed, is large enough to occlude the coronary sinus 352 and the MCV 354 when abutted to the ostium of the coronary sinus 352. In at least one embodiment, a diameter of the stent structure 1010 is from about 10 mm to about 30 mm.

### Perfusion Catheter Embodiments

Embodiments of exemplary catheters for use as perfusion catheters in an LRP system are now described. In at least one embodiment, the perfusion catheters are designed to support a liquid perfusion flow rate of 150 mL/min or greater (e.g., about 700 mL/min or greater). In embodiments that utilize multiple perfusion catheters (e.g., insertion of a first catheter into the right coronary artery and insertion of a second catheter into the left coronary artery) can support a combined flow capacity of 700 mL/min or greater.

Certain embodiments of the recovery catheters are advantageous for use in the supply line of an LRP system used to form a closed-circuit within an unarrested beating heart when inserted into the coronary arteries. The catheters described herein can be designed to satisfy the following criteria: capability of femoral access to the coronary arteries; an outer diameter for coronary artery entry of 8 Fr or less; an outer diameter for occlusion of about 6 mm to about 8 mm; compatibility with a 0.018-inch guidewire and a 0.014-inch pressure wire; and the ability to maintain stable position for at least 60 minutes during an LRP procedure.

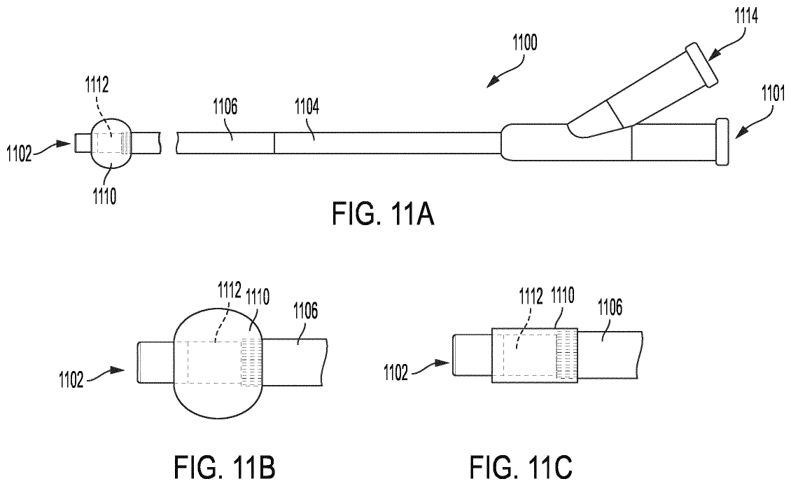

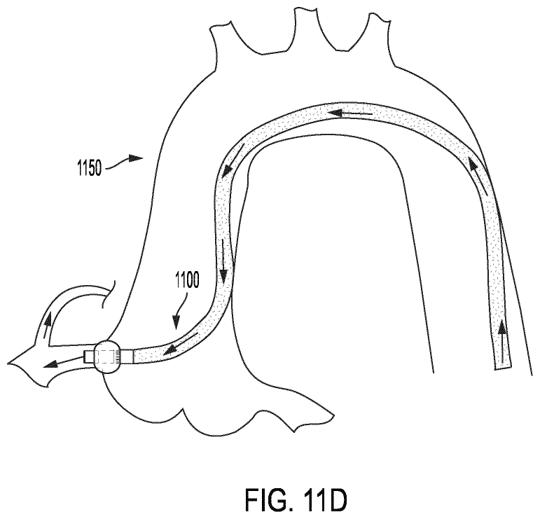

FIGS. 11-16 depict various catheter embodiments suitable for fluid perfusion in an LRP system. Any of the catheters depicted in FIGS. 11-16 may be configured to support liquid flow rates (suction or perfusion) of at least about 150 mL/min, at least about 200 mL/min, at least about 200 mL/min, at least about 250 mL/min, at least about 300 mL/min, at least about 350 mL/min, at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, at least about 1000 mL/min, or within any range defined by any of the foregoing lower limits (e.g., from about 150 mL/min to about 1000 mL/min, from about 400 mL/min to about 800 mL/min, etc.). Each catheter can be designed to have a smooth profile from a proximal catheter body to a low distal profile, for example, using one or more concentric lumen shafts. In addition, the catheters can be designed to have lumen shafts that are pre-shaped depending on the anatomy in which the LRP procedure is to be performed, which may improve overall stability during use. Examples of pre-shaped catheter lumens are illustrated in FIG. 17.

In certain embodiments, one or more of the catheters may be multi-lumen catheters, such as double-lumen catheters. In certain embodiments, the multi-lumen catheters allow for liquid flow (e.g., a perfusate) and enable inflation of one or more balloons. In certain embodiments, one or more of the catheters may be multi-balloon catheters having two or more balloons. In certain embodiments, one or more of the balloons may be deployed or deflated independently.

FIGS. 11A-11C illustrate an exemplary catheter 1100 having a lumen shaft 1104/1106 with a proximal end 1101 and a distal end 1102 having an opening from which a perfusate can flow. The lumen shaft 1104/1106 can be formed from an outer lumen shaft 1104 that at least partially encompasses an inner lumen shaft 1106 to expose a distal portion of the inner lumen shaft 1106 near the distal end 1102. The proximal end 1101 includes an outlet structure that can be fluidly coupled to an LRP system. One or more of the outer lumen shaft 1104 or the inner lumen shaft 1106 may be formed from a durable polymer material such as a polyether block amide (PEBA) material (e.g., commercially available as PEBAX®). In at least one embodiment, an innermost diameter of the inner lumen shaft 1106 is at least about 2 mm, at least about 2.5 mm, at least about 3 mm, at least about 3.5 mm, at least about 4 mm, at least about 4.5 mm, or at least about 5 mm to provide a liquid flow path.

The catheter 1100 includes an expandable balloon structure 1110 disposed along a portion 1112 corresponding to the inner lumen shaft 1106 and a tip portion formed by an additional lumen. In at least one embodiment, the inner lumen shaft 1106 includes a concentric inner flow path surrounding the liquid flow path. The concentric inner flow path provides a path for gas flow from the balloon structure 1110 to a port 1114, which can be used to inflate or deflate the balloon depending on the pressure applied at the port 1114. In at least one embodiment, an outermost surface of the inner lumen shaft 1106 at the portion 1112 is removed such that the portion 1112 is sealed by the balloon structure 1110 to isolate gas flow from the concentric inner flow path to the balloon structure 1110. In at least one embodiment, an expanded diameter of the balloon structure is from about 15 mm to about 30 mm, about 15 mm to about 20 mm, about 20 mm to about 25 mm, about 24 mm to about 28 mm, about 25 mm to about 30 mm, or within any subrange defined therebetween (e.g., about 20 mm to about 28 mm). FIGS. 11B and 11C illustrate the balloon in its deployed and deflated states, respectively.

FIG. 11D illustrates deployment of the catheter 1100 in an aorta 1150 in accordance with at least one embodiment. As shown, the catheter 1100 is pre-shaped for insertion into the aorta 1150 for ease of navigation. Moreover, the shape can leverage back-up forces from the aortic wall to further enhance stability during occlusion and perfusion of the coronary artery.

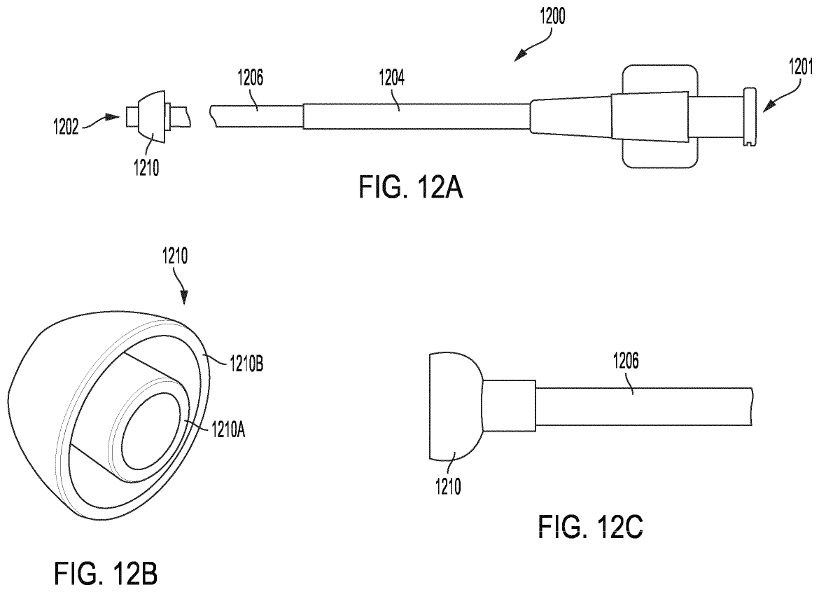

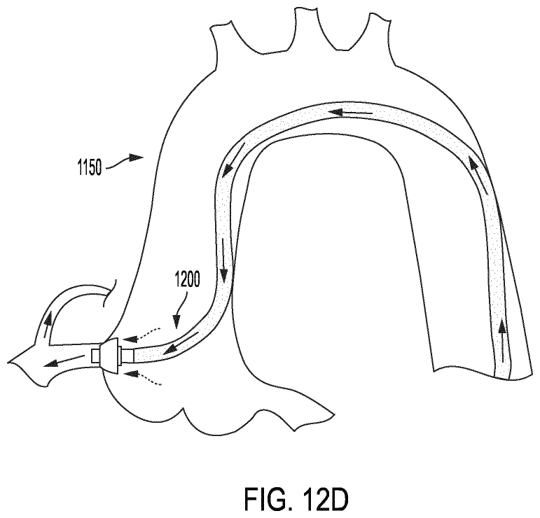

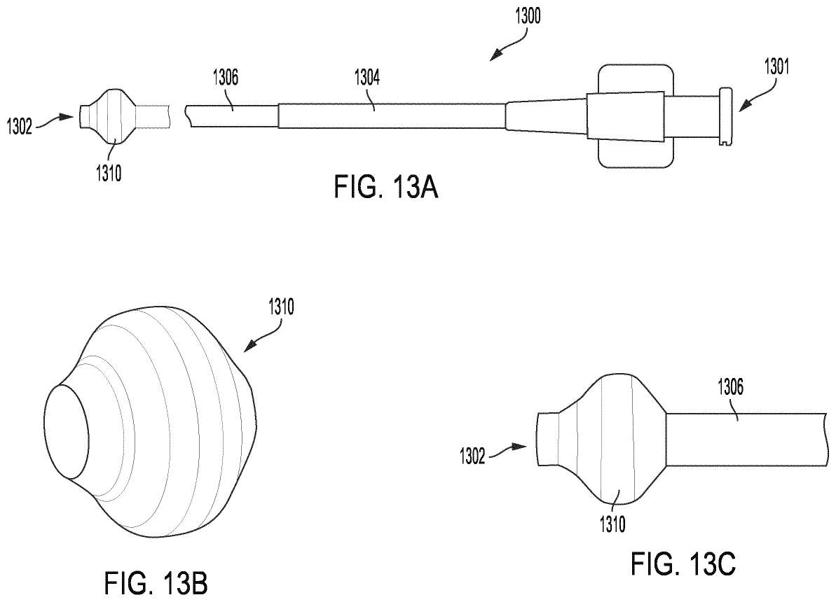

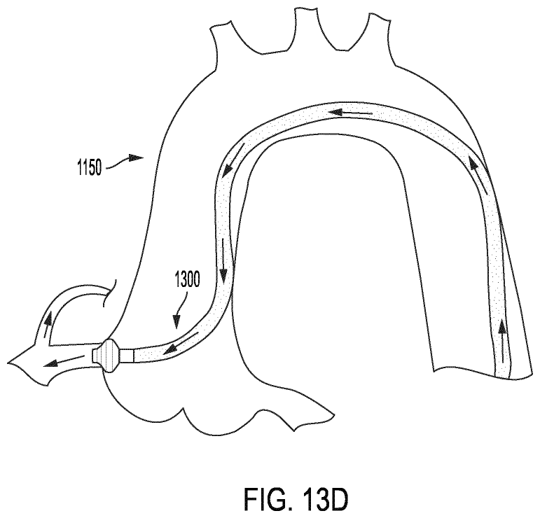

FIGS. 12 and 13 illustrate catheters that include plug and wedge occlusion structures, respectively, that advantageously adapt their shapes to a vessel or ostium, are formed from highly compressible and atraumatic materials for safe introduction and deployment, are shorter in length in comparison to a balloon structure, and do not require an additional lumen for inflation as would a balloon structure.

FIGS. 12A-12C illustrate an exemplary catheter 1200 having a lumen shaft 1204/1206 with a proximal end 1201 and a distal end 1202 having an opening from which a perfusate can flow. The lumen shaft 1204/1206 can be formed from an outer lumen shaft 1204 that at least partially encompasses an inner lumen shaft 1206 to expose a distal portion of the inner lumen shaft 1206 near the distal end 1202. The proximal end 1201 includes an outlet structure that can be fluidly coupled to an LRP system. One or more of the outer lumen shaft 1204 or the inner lumen shaft 1206 may be formed from a durable polymer material such as a polyether block amide (PEBA) material (e.g., commercially available as PEBAX®). In at least one embodiment, an innermost diameter of the inner lumen shaft 1206 is at least about 2 mm, at least about 2.5 mm, at least about 3 mm, at least about 3.5 mm, at least about 4 mm, at least about 4.5 mm, or at least about 5 mm to provide a liquid flow path.

The catheter 1200 further includes a plug 1210 near the distal end 1202. In at least one embodiment, the plug 1210 is formed from a flexible material, such as silicone or a foam material. In at least one embodiment, the plug 1210 includes an inner portion 1210A that fits onto the inner lumen shaft 1206 and a flexible outer portion 1210B shaped to be configurable between a retracted state (FIG. 12A) and an extended state (FIG. 12C) for which the outer portion 1210B extends distally from the distal end 1202. The plug 1210 in FIG. 12A is illustrated as tapering in a distal direction. In at least one embodiment, the plug 1210 may be reversed such that it tapers in a proximal direction. In at least one embodiment, the outer lumen 1204 may be configured to cover the plug 1210 prior to deployment.

FIG. 12D illustrates deployment of the catheter 1200 in an aorta 1150 in accordance with at least one embodiment. The pressure of the arterial blood flow into the hollow space between the inner portion 1210A and the outer portion 1210B of the plug 1210 can help improve the sealing of the catheter 1200 within the coronary artery. As shown, the catheter 1200 is pre-shaped for insertion into the aorta 1150 for ease of navigation. Moreover, the shape can leverage back-up forces from the aortic wall to further enhance stability during occlusion and perfusion of the coronary artery.

FIGS. 13A-13C illustrate an exemplary catheter 1300 having a lumen shaft 1304/1306 with a proximal end 1301 and a distal end 1302 having an opening from which a perfusate can flow. The lumen shaft 1304/1306 can be formed from an outer lumen shaft 1304 that at least partially encompasses an inner lumen shaft 1306 to expose a distal portion of the inner lumen shaft 1306 near the distal end 1302. The proximal end 1301 includes an outlet structure that can be fluidly coupled to an LRP system. One or more of the outer lumen shaft 1304 or the inner lumen shaft 1306 may be formed from a durable polymer material such as a polyether block amide (PEBA) material (e.g., commercially available as PEBAX®). In at least one embodiment, an innermost diameter of the inner lumen shaft 1306 is at least about 2 mm, at least about 2.5 mm, at least about 3 mm, at least about 3.5 mm, at least about 4 mm, at least about 4.5 mm, or at least about 5 mm to provide a liquid flow path.

The catheter 1300 further includes a wedge 1310 near the distal end 1302, which may be shaped to adapt to a vessel or ostium. In at least one embodiment, the wedge 1310 is formed from a flexible material, such as silicone or a foam material. In at least one embodiment, the outer lumen shaft 1304 may be configured to cover the wedge 1310 prior to deployment.

FIG. 13D illustrates deployment of the catheter 1300 in an aorta 1150 in accordance with at least one embodiment. As shown, the catheter 1300 is pre-shaped for insertion into the aorta 1150 for ease of navigation. Moreover, the shape can leverage back-up forces from the aortic wall to further enhance stability during occlusion and perfusion of the coronary artery.

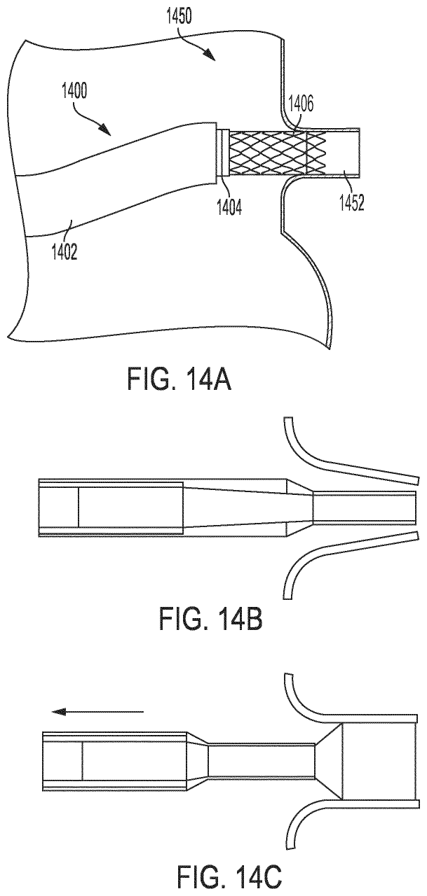

FIGS. 14A-14C illustrate an exemplary catheter 1400 that includes a partially covered and recapturable stent structure 1406 in accordance with at least one embodiment, similar to the catheter 800 described with respect to FIG. 8. The catheter 1400 is illustrated as being inserted into a coronary artery 1452 via the aorta 1450. The catheter 1400 includes an outer lumen shaft 1402 and an inner lumen shaft 1404 that is coupled to the stent structure 1406 in certain embodiments. The stent structure 1406 is further depicted as having a proximal covered portion, which may be formed from a flexible and durable polymer material, and an distal uncovered portion. FIGS. 14B and 14C illustrate placement and deployment, respectively, of the stent structure 1406 when inserted into the coronary artery 1452. Deployment of the stent structure 1406 is performed by moving the outer lumen shaft 1402 in the proximal direction.

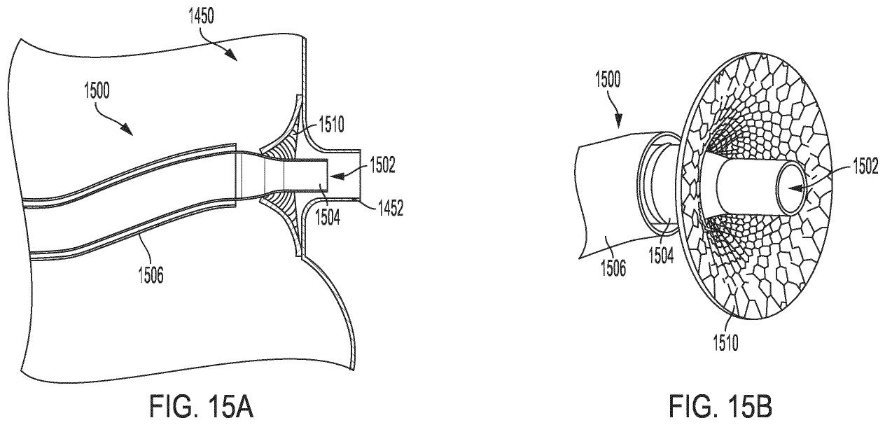

FIGS. 15A and 15B illustrate an exemplary catheter 1500 that includes a releasable covered braided disk 1510 in accordance with at least one embodiment. The catheter 1500 includes an outer lumen shaft 1506 and an inner lumen shaft 1504. The braided disk 1510 is contained within the outer lumen shaft 1506 during placement of the catheter 1500, and can be deployed by moving the outer lumen shaft 1506 in the proximal direction. In certain embodiments, when deployed, the braided disk 1510 does not expand past the distal end 1502, and is used to stabilize the catheter 1500 against the ostium of the coronary artery 1452 to reduce the risk of stenosis during occlusion the coronary artery 1452, while allowing the distal end 1502 to extend into the coronary artery 1452.

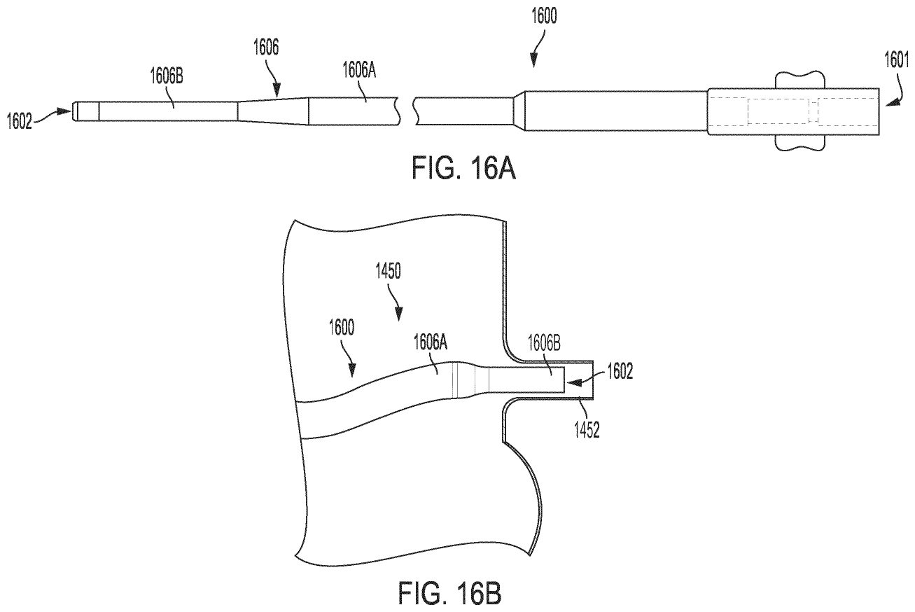

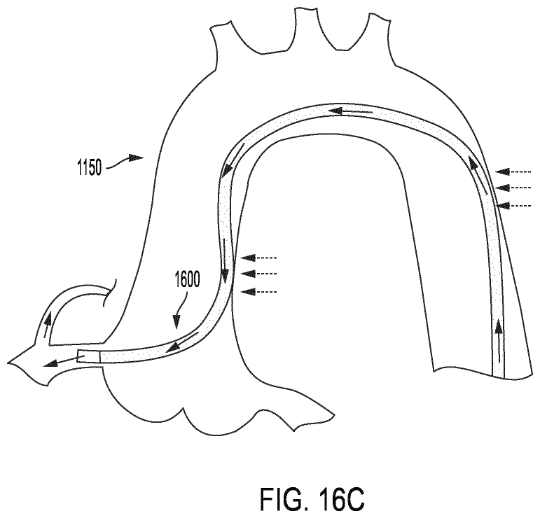

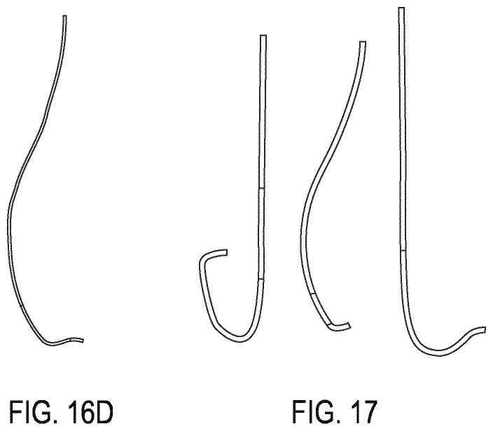

FIGS. 16A-16D illustrate an exemplary catheter 1600 having a lumen shaft 1606 with a proximal end 1601 and a distal end 1602 having an opening from which a perfusate can flow. The proximal end 1601 includes an outlet structure that can be fluidly coupled to an LRP system. The lumen shaft 1604 may be formed from a durable polymer material such as a polyether block amide (PEBA) material (e.g., commercially available as PEBAX®). In at least one embodiment, an innermost diameter of the lumen shaft 1606 is at least about 2 mm, at least about 2.5 mm, at least about 3 mm, at least about 3.5 mm, at least about 4 mm, at least about 4.5 mm, or at least about 5 mm to provide a liquid flow path. In at least one embodiment, a proximal portion 1606A of the lumen shaft 1606 may have a larger diameter than a distal portion 1606B of the lumen shaft 1606, and can taper gradually over a length of the lumen shaft 1606.

FIG. 16C illustrates deployment of the catheter 1600 in an aorta 1150 in accordance with at least one embodiment. As shown, the catheter 1600 is pre-shaped for insertion into the aorta 1150 for ease of navigation. Moreover, the shape can leverage back-up forces from the aortic wall to further enhance stability during occlusion and perfusion of the coronary artery.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A recovery catheter comprising:
a lumen shaft having a proximal end and a distal end;
an expandable balloon structure disposed near the distal end of the lumen shaft; and
a tip portion between the expandable balloon structure and the distal end of the lumen shaft,
wherein the recovery catheter is configured to support a liquid flow rate of about 400 mL/min or greater.

2. The recovery catheter of claim 1, wherein the tip portion comprises an elongated shaft extending from the expandable balloon structure to the distal end of the lumen shaft, and
wherein the tip portion comprises a distal opening at the distal end and a plurality of perforations along the elongated shaft.

3. (canceled)

4. The recovery catheter of claim 2, wherein the length of the elongated shaft of the tip portion is from about 2 mm to about 35 mm.

5. The recovery catheter of claim 1, wherein an inner diameter of the lumen shaft is at least about 2 mm.

6. The recovery catheter of claim 1, wherein an expanded diameter of the expandable balloon structure is from about 15 mm to about 30 mm.

7. The recovery catheter of claim 1, wherein the lumen shaft is a multi-lumen shaft comprising at least two lumen shafts.

8. The recovery catheter of claim 7, wherein the multi-lumen shaft comprises an inner lumen shaft and an outer lumen shaft that at least partially encompasses the inner lumen shaft to expose a proximal portion of the inner lumen shaft near the proximal end of the lumen shaft, and wherein the expandable balloon structure and the tip portion are disposed on the inner lumen shaft.

9. The recovery catheter of claim 1, wherein the recovery catheter is a multi-balloon catheter further comprising at least an additional expandable balloon structure disposed adjacent to the expandable balloon structure.

10. The recovery catheter of claim 9, wherein an expanded diameter of the additional expandable balloon structure is greater than an expanded diameter of the expandable balloon structure.

11. The recovery catheter of claim 9, wherein a portion of the lumen shaft between the expandable balloon structure and the additional expandable balloon structure comprises one or more perforations.

12. The recovery catheter of claim 1, further comprising:
a stent structure disposed on a portion of the lumen shaft between the tip portion and the expandable balloon structure; and
perforations along the lumen shaft between the expandable balloon structure and the stent structure and/or between the stent structure and the distal end.

13. (canceled)

14. A recovery catheter comprising:
a lumen shaft having a proximal end and a distal end;
a covered disk-shaped stent structure disposed near the distal end of the lumen shaft; and
a tip portion between the covered disk-shaped stent structure and the distal end of the lumen shaft,
wherein the recovery catheter is configured to support a liquid flow rate of about 400 mL/min or greater.

15. The recovery catheter of claim 14, wherein the tip portion comprises a radio marker or a radiopaque filler composition.

16. A recovery catheter comprising:
a lumen shaft having a proximal end and a distal end; and
a deployable stent structure disposed at the distal end of the lumen shaft, the stent structure comprising a covering on a proximal portion, wherein the stent structure, when deployed, is uncovered at a distal portion,
wherein the recovery catheter is configured to support a liquid flow rate of about 400 mL/min or greater.

17. The recovery catheter of claim 16, wherein the recovery catheter is configured to support a liquid flow rate of at least about 1000 mL/min.

18. A perfusion catheter comprising:
a lumen shaft having a proximal end and a distal end;
an occlusion structure near the distal end of the lumen shaft, the occlusion structure adapted to seal perfused blood from systemic blood circulation when inserted into a vascular blood vessel, the occlusion structure being selected from:
a balloon,
a plug configurable between a retracted state and an extended state;
a wedge shaped to adapt to a vessel or ostium;
a releasable covered stent;
a releasable braided disk; and
a tip portion comprising an elongated shaft extending from the occlusion structure to the proximal end of the lumen shaft, the tip portion comprising a proximal opening at the proximal end,
wherein the perfusion catheter is configured to support a liquid flow rate of about 400 mL/min or greater.

19. The perfusion catheter of claim 18, wherein the occlusion structure comprises the plug configurable between a retracted state and an extended state, wherein when in the extended state, a portion of the plug extends distally from the tip portion.

20. The perfusion catheter of claim 18, wherein the occlusion structure comprises the releasable covered stent, wherein the lumen shaft comprises an outer sheath that covers the releasable covered stent, and wherein, when the sheath is retracted proximally, the releasable covered stent is deployed past the distal end of the lumen shaft having an expanded diameter that is greater than the diameter of the lumen shaft.

21. The perfusion catheter of claim 18, wherein the occlusion structure comprises the releasable braided disk, wherein the lumen shaft comprises an outer sheath that covers the releasable braided disk, wherein, when the sheath is retracted proximally, the releasable braided disk is deployed having an expanded diameter that is greater than the diameter of the lumen shaft, and wherein the tip portion extends distally past the releasable braided disk when deployed.

22. The perfusion catheter of claim 18, wherein the perfusion catheter is configured to support a liquid flow rate of at least about 1000 mL/min.
